(19) United States
(12) Patent Application Publication
(10) Pub. No.: US 2022/0202890 A1
(43) Pub. Date: Jun. 30, 2022

(54) FORMULATION CONTAINING CORNUS WILSONIANA EXTRACT AND USE THEREOF

(71) Applicant: The Second Xiangya Hospital, Central South University, Hunan (CN)

(72) Inventors: Xundi XU, Hunan (CN); Qiang LIU, Hunan (CN)

(21) Appl. No.: 17/595,060

(22) PCT Filed: May 8, 2020
(86) PCT No.: PCT/CN2020/089246

§ 371 Date: Nov. 8, 2021

(30) Foreign Application Priority Data
May 8, 2019 (CN) 201910378567.5

(51) Int. Cl. A61K 36/40; A61P 1/16; A23L 33/00; A23L 33/105

(52) U.S. Cl. CPC A61K 36/40; A61P 1/16; A23V 2002/00; A23L 33/105; A23L 33/40

(57) ABSTRACT

The present invention provides a formulation containing an extract from Cornus wilsoniana and use thereof, and the extract from Cornus wilsoniana is for example the Cornus wilsoniana oil. The formulation containing the extract from Cornus wilsoniana provided by the present invention can effectively improve the condition of hepatic fibrosis, thus providing a new approach for treating hepatic fibrosis.

### CROSS-REFERENCE TO RELATED APPLICATION(S)

This application is a national phase application of International Patent Application No. PCT/CN2020/089246 (published as WO 2020/224649), filed May 8, 2020, which claims priority to CN Application Serial No. 201910378567.5 filed May 8, 2019, both of which are hereby incorporated by reference in their entireties.

### TECHNICAL FIELD

The present invention belongs to the field of formulations, and particularly relates to use of an extract from Cornus wilsoniana.

### BACKGROUND

The incidence of chronic liver diseases is relatively high in China. Hepatic fibrosis is a link in the pathological development of many chronic hepatic diseases, such as viral hepatitis, hepatic injury caused by toxins, alcoholic hepatitis, nonalcoholic hepatitis, etc. The development of hepatic fibrosis may lead to hepatic cirrhosis, portal hypertension, decompensation of hepatic function, liver tumor and even death. Every year, more than one million people die from this disease, especially for end-stage liver diseases, there is no effective reversal agent available. Liver transplantation is the most effective method; however, many patients die during the process of waiting for liver transplantation due to insufficient donors. Therefore, there is an urgent need to research and develop medicaments for this kind of diseases.

### SUMMARY

The present inventors has studied the anti-fibrosis effect of an extract from Cornus wilsoniana, after verified by experiments, the present inventors propose a new way to improve hepatic fibrosis.

In a first aspect, the present invention provides an anti-hepatic fibrosis formulation, which comprises an extract from Cornus wilsoniana.

In a second aspect, the present invention provides use of an extract from Cornus wilsoniana in preparing an anti-hepatic fibrosis formulation.

Preferably, the extract from Cornus wilsoniana is Cornus wilsoniana oil. More preferably, the Cornus wilsoniana oil is a lipid substance extracted from the fruits and seeds of Cornus wilsoniana.

Preferably, the hepatic fibrosis is induced by CCl₄ (carbon tetrachloride).

Preferably, the formulation is a food, a medicament or a health care product.

Preferably, when the formulation is a medicament, the formulation is administered by oral administration, injection or intragastric administration. Further preferably, the injection is intravenous injection or intraperitoneal injection.

Preferably, the medicament is in a form of tablet, capsule, powder-injection, injection or aerosol.

Preferably, the medicament further comprises one or more pharmaceutically acceptable excipients, such as diluents, adhesives, wetting agents, disintegrating agents, solvents and/or buffers, etc. In order to prepare suitable medicaments, a person skilled in the art can select appropriate type and amount of excipients according to actual needs.

Preferably, when the formulation is a food or a health care product, the formulation further comprises one or more excipients acceptable in the field of galenic pharmacy, such as fillers, taste improvers, solvents and/or buffers, etc. In order to prepare a suitable food or health care product, a person skilled in the art can choose appropriate type and amount of excipients according to actual needs

In a third aspect, the present invention provides a method for anti-hepatic fibrosis, which comprises a step of administering the extract from Cornus wilsoniana or formulation thereof according to the present invention to an animal (such as human) in need thereof.

In a fourth aspect, the present invention provides use of the extract from Cornus wilsoniana or formulation thereof according to the present invention in anti-hepatic fibrosis.

Preferably, the anti-hepatic fibrosis in the present invention refers to preventing or treating hepatic fibrosis or improving the condition of hepatic fibrosis.

The formulation containing Cornus wilsoniana oil provided by the present invention can effectively improve the condition of hepatic fibrosis, thus providing a new way for treating hepatic fibrosis.

### BRIEF DESCRIPTION OF THE DRAWING

FIGS. 1A-1F show test results of hepatic function indexes and blood lipid indexes in Example 2, wherein FIG. 1A is an ALT test result, FIG. 1B is an AST test result, and both of them are hepatic function indexes; FIG. 1C is a TG test result, FIG. 1D is a TC test result, FIG. 1E is a HDL-C test result, FIG. 1F is a LDL-C test result, and all of the above four are blood lipid indexes; wherein, * means P<0.05; ** means P<0.01.

FIGS. 2A-2C show test results of serum hepatic fibrosis indexes in Example 2, wherein FIG. 2A is a PCIII test result, FIG. 2B is a IV-C test result, and FIG. 2C is a LN test result.

FIGS. 3A-3C show test results of indexes of antioxidation capability in Example 2, wherein FIG. 3A is a SOD test result, FIG. 3B is a MDA test result, and FIG. 3C is a GSH test result.

FIG. 4 shows results of HE staining, Sirius red staining and Masson staining of liver tissue.

FIGS. 5A-5B show Western blot results in Example 2, wherein FIG. 5A is an α-SMA test result, and FIG. 5B is a TGF-β1 test result.

### EMBODIMENTS

The contents of the present invention will be illustrated below in combination with specific examples, but the scope of the present invention is not limited thereto. Any technical solutions based on the inventive concept of the present invention falls within the scope of the present invention.

Unless otherwise specified, any reagents and instruments used in the following examples are conventional reagents and instruments in the field and are commercially available. Any methods used in the present invention are conventional methods in the field, and a person skilled in the art can undoubtedly implement this method and obtain corresponding results according to the contents of the examples.

Cornus wilsoniana oil is a lipid substance extracted from the fruits and seeds of Cornus wilsoniana (Swida wilsoniana), which mainly contains active ingredients such as oleic acid and linoleic acid. Cornus wilsoniana oil can treat hyperlipidemia (hypertension and coronary heart disease) and has a significant effect on lowering cholesterol, wherein the unsaturated fatty acids have antioxidant effect and can decrease the damage of lipid peroxidation. However, the anti-hepatic fibrosis effect of the Cornus wilsoniana oil has not been reported yet. In order to observe the anti-hepatic fibrosis effect of Cornus wilsoniana oil, the following examples will study the therapeutic effect of the Cornus wilsoniana oil on hepatic fibrosis using animal models with hepatic fibrosis induced by CCl₄.

The mice used in the following examples were C57BL/6J mice provided by Hunan SJA Laboratory Animal Co., Ltd., and with a license number of SCXK (Xiang) 2016-0002. The mice were raised in SPF laboratory.

The Cornus wilsoniana oil was from Hunan Academy of Forestry. The Cornus wilsoniana oil fatty acid mainly comprised oleic acid (35.71%) and linoleic acid (44.49%), and both oleic acid and linoleic acid are unsaturated fatty acids. The Cornus wilsoniana oil fatty acid further contains other beneficial components, which are phytosterol (1.98 mg/g), squalene (0.0324 mg/g) and vitamin E (0.60556 mg/g).

CCl₄ (No.: C112040) and olive oil (No.: O108686) were purchased from Aladdin Company. HE staining kit (No.: G1120), Sirius Red staining kit (No.: G1471) and Masson trichrome staining kit (No.: G1340) were all purchased from Solarbio Company. β-actin (No.: 60008.I.AP) was purchased from Proteintech Company, and α-SMA (No.: ab32575) and TGFβ1 (No.: ab215715) were purchased from Abcam Company.

### Example 1: Experiment Animal Grouping and Treatment

The mice were selected and subjected to clean grade feeding. After an adaptation period of one week, the mice were randomly divided into four groups, namely:

normal group (NC): olive oil control group, 7 mice;

model group (Model): CCl₄ group, 5 mice;

low-dose Cornus wilsoniana oil group (LDG): a group of CCl₄+low-dose Cornus wilsoniana oil (0.5 mg/kg), 6 mice;

high-dose Cornus wilsoniana oil group (HDG): a group of CCl₄+high-dose Cornus wilsoniana oil (2 mg/kg), 6 mice.

The mice in the last three groups were given CCl₄ administration in the following manner: CCl₄ was dissolved in olive oil at a concentration of 20%, and injected intraperitoneally twice a week with a dose of 5 mL/kg. The mice in the normal group were injected with the same amount of olive oil. The Cornus wilsoniana oil was given to the mice by intragastrical administration every day with the corresponding dose. After 6 weeks of continuous administration, the mice were killed, and serum and liver samples were collected.

### Example 2: Test Items

The serum and liver samples collected in Example 1 were tested, and the test items and methods were as follows:

Hepatic function indexes and blood lipid indexes were tested by the clinical laboratory of Xiangya Second Hospital of Central South University.

Serum hepatic fibrosis indexes were tested by the Laboratory of Infectious Diseases Department of Xiangya Second Hospital of Central South University.

Antioxidant indexes were tested by the kit provided by Nanjing Jiancheng Institute of Bioengineering according to the instructions.

The liver sections of mice were stained according to the instructions of the corresponding staining kit.

Western blot was used to test protein expression level.

The test results were shown in Table 1, and the details were described as follows:

2.1 Hepatic Function Indexes

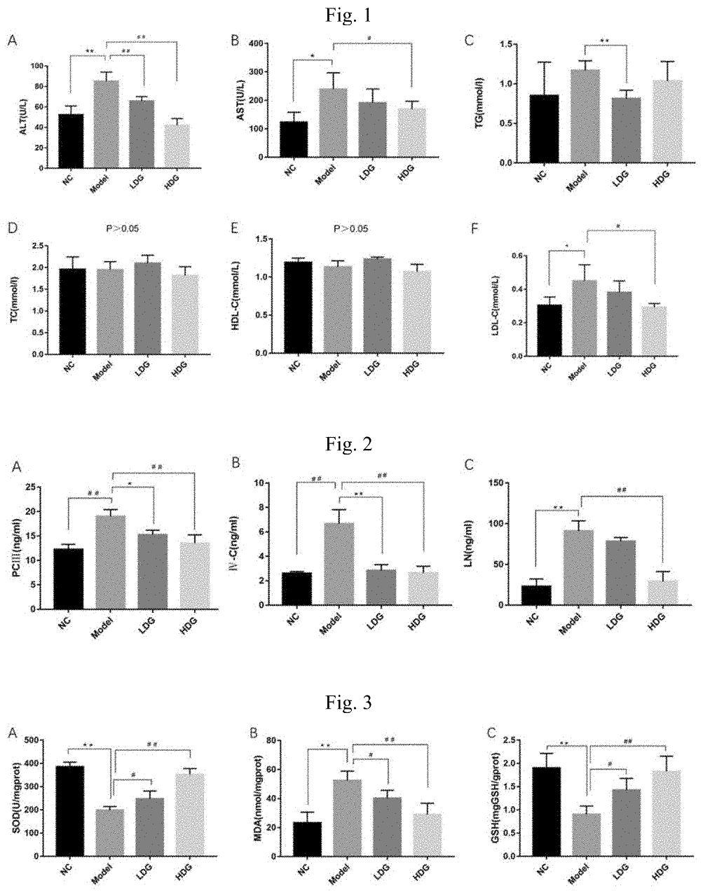

FIGS. 1A and 1B showed the test results of hepatic function indexes: glutamic-pyruvic transaminase (ALT) and glutamic oxalacetic transaminase (AST). It can be seen from the figures that the difference between the contents of ALT and AST in the mice in the model group and those in the mice in the normal group were statistically significant (P<0.05), indicating that the model was successfully established; the contents of serum ALT and AST in the mice in the low-dose Cornus wilsoniana oil group and the high-dose Cornus wilsoniana oil group were lower than those in the mice in the model group, and the differences were statistically significant (P<0.05). The results show that the Cornus wilsoniana oil can protect hepatic function.

2.2 Blood Lipid Indexes

The test results of blood lipid indexes were given by FIGS. 1C-1F of FIG. 1, wherein, compared with the normal group, the contents of triglyceride (TG), total cholesterol (TC) and high density lipoprotein (HDL-C) in the mice in the model group, the low-dose Cornus wilsoniana oil group and the high-dose Cornus wilsoniana oil group were not significantly different (P>0.05), as shown in FIGS. 1C, 1D and 1E. With regard to low density lipoprotein (LDL-C), the contents in the mice in the model group were higher than those in the mice in the normal group, and the differences of LDL-C contents between the mice in the high-dose Cornus wilsoniana oil group and the mice in the model group were statistically significant (P<0.05), as shown in FIG. 1F, indicating that high-dose Cornus wilsoniana oil can reduce serum LDL, which is beneficial for improving blood lipid conditions.

2.3 Serum Hepatic Fibrosis Indexes

Serum hepatic fibrosis indexes comprise type III procollagen (PCIII), laminin (LN) and type IV collagen (IV-C). As shown in FIGS. 2A, 2B and 2C, the contents of PCIII, IV-C and LN in the serum of the mice in the model group were significantly higher than those in the mice in the normal group (P<0.05). The low-dose Cornus wilsoniana oil group could reduce the contents of PCIII and IV-C in the serum of the mice to a certain extent (P<0.01). The high-dose Cornus wilsoniana oil group could reduce the contents of serum PCIII, IV-C and LN in the mice significantly (P<0.01). The results show that the Cornus wilsoniana oil can reduce hepatic fibrosis-related serum indexes, indicating that the Cornus wilsoniana oil can alleviate hepatic fibrosis conditions induced by CCl₄ in mice.

2.4 Indexes of Antioxidation Capability

Indexes of antioxidation capability comprise superoxide dismutase (SOD), malondialdehyde (MDA) and glutathione (GSH). It can be seen from FIG. 3B that the contents of liver MDA of the mice in the model group were higher than those in the mice in the normal group, while the contents of MDA in the mice in the high-dose Cornus wilsoniana oil group and the low-dose Cornus wilsoniana oil group were lower than those in the mice in the model group (P<0.05). It can be seen from FIGS. 3A and 3C that the contents of liver SOD and GSH in the mice in the model group were lower than those in the mice in the normal group, while the contents of SOD and GSH in the mice in the high-dose Cornus wilsoniana oil group and the low-dose Cornus wilsoniana oil group were higher than those in the mice in the model group (P<0.05). The results show that hepatic fibrosis of mice induced by CCl₄ can reduce the antioxidation capability of the liver in mice, while the Cornus wilsoniana oil can enhance the antioxidation capability of the liver in the mice suffered from hepatic fibrosis.

2.5 HE Staining of Mice Liver

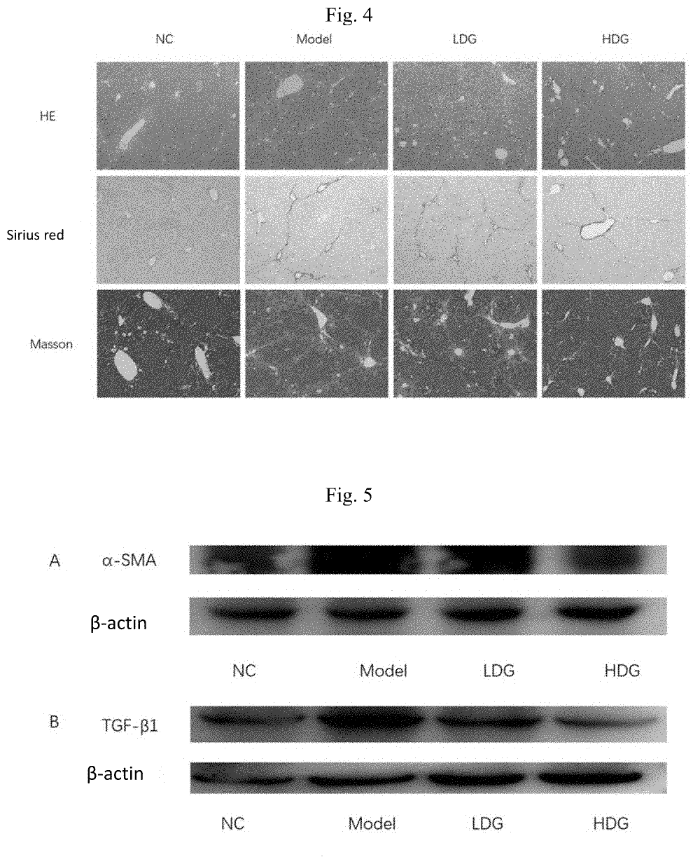

The liver tissues of mice were stained with HE according to the routine procedure, and the results were shown in the first line of FIG. 4, wherein, in the normal group, the liver cells were arranged radially with the central vein being the center, and the structure of the hepatic lobule was normal and there was no hepatocytic degeneration and inflammatory cell infiltration; in the model group, the hepatic cords were disordered, it can be seen that obvious fibrous septa were formed, collagen had significant hyperplasia, and hepatocytes were markedly swollen and degenerated, most of which were steatosis and distributed around the boundary plate, and inflammatory cell infiltration can be seen in the interstitium; in the high-dose Cornus wilsoniana oil group, the structure of hepatic lobule, a small amount of fibrous septa, a small amount of hepatocyte steatosis and mild inflammatory cells infiltration were observed; in the low-dose Cornus wilsoniana oil group fibrous septa were formed, collagen hyperplasia was obvious, and hepatocytes swelled and moderate inflammatory infiltration can be seen in the interstitium.

It can be seen from FIG. 4 that Cornus wilsoniana oil can improve the damage of liver cells in mice and alleviate liver inflammation at the same time.

2.6 Sirius Red Staining and Masson Staining of Mice Liver

The liver tissues of mice were examined microscopically after Sirius red and Masson staining, and the results were shown in the second and third lines of FIG. 4. In the normal group, only a small amount of collagen was observed in the portal area. In the model group, collagen hyperplasia was obvious, complete fibrous septa were formed, and the normal hepatic lobule structure was destroyed. In the high-dose Cornus wilsoniana oil group, a small amount of collagen hyperplasia was observed and a small amount of fibrous septa were formed indicating that the Cornus wilsoniana oil can alleviate the formation of hepatic fibrosis. In the low-dose Cornus wilsoniana oil group, it can be seen that collagen hyperplasia was obvious and incomplete fibrous septa were formed, and the structure of hepatic lobule was slightly damaged.

2.7 Protein Expression Levels of α-SMA (α-Smooth Muscle Actin) and TGF-β1 (Transforming Growth Factor β1) in Mice Liver

TGF-β is an important regulatory factor of hepatic fibrosis, thus also a biomarker of hepatic fibrosis. TGF-β is located in cytoplasm. Up-regulation of α-SMA expression is a characteristic manifestation of the activation of hepatic stellate cell, thus also a biomarker of hepatic fibrosis. α-SMA is located in fibroblasts and smooth muscle cells.

The liver proteins of mice in each group were extracted, and Western blot experiment was carried out. The results showed that the expression levels of α-SMA and TGF-β1 protein in the liver of the mice in the model group were higher than those in the mice in the normal group, and the difference was significant. The expression levels of α-SMA and TGF-β1 protein in the livers in the low-dose Cornus wilsoniana oil group were slightly lower than those in the livers in the model group. Compared with the model group, the expression levels of α-SMA and TGF-β1 protein in the liver in the high-dose Cornus wilsoniana oil group were significantly lower, and the difference was significant (see FIGS. 5A and 5B).

The results show that the Cornus wilsoniana oil can decrease the hepatic fibrosis indexes α-SMA in mice in a dose-dependent manner, and can also decrease the factor TGF-β1 which promotes the formation of hepatic fibrosis in a dose-dependent manner.

TABLE 1

| | | | | | | | | | | | | |
|---|---|---|---|---|---|---|---|---|---|---|---|---|
| | | | | | | | Type III | | | | | |
| | | | | | | | procollagen | | | SOD | MDA | GSH |
| Group | ALT | AST | TG | TC | HDL-C | LDL-C | N-terminal | Type IV | Laminin | (U/ | (mmol/ | (mgGSH/ |
| of mice | (u/l) | (u/l) | (mmol/l) | (mmol/L) | (mmol/L) | (mmol/L) | peptide | collagen | (LN) | mgprot) | mgprot) | gprot) |

Normal group

| | | | | | | | | | | | | |
|---|---|---|---|---|---|---|---|---|---|---|---|---|
| 1 | 52.2 | 120.6 | 0.55 | 2 | 1.37 | 0.24 | 11.4 | 2.1 | 22.5 | 342.03 | 27.35 | 1.49 |
| 2 | 35.9 | 82.6 | 1.58 | 2 | 1.22 | 0.29 | 10.5 | 2.9 | 38.8 | 414.33 | 15.98 | 2.59 |
| 3 | 55.2 | 112.5 | 1.68 | 2.2 | 1.16 | 0.36 | 14.6 | 2.6 | 26.2 | 317.92 | 17.34 | 1.92 |
| 4 | 49.5 | 109.7 | 0.85 | 1.7 | 1.15 | 0.35 | 11.2 | 2.5 | 26.8 | 349.12 | 20.19 | 1.95 |
| 5 | 46.4 | 89.6 | 0.52 | 1.44 | 0.96 | 0.2 | 14.2 | 2.4 | 23.6 | 311.20 | 24.81 | 1.68 |
| 6 | 40.1 | 120.9 | 0.68 | 2.16 | 1.28 | 0.28 | 13.4 | 2.7 | 18.8 | 420.52 | 18.29 | 1.98 |
| 7 | 42.9 | 108.5 | 0.6 | 2.2 | 1.15 | 0.45 | 10.6 | 2.5 | 23.8 | 350.26 | 21.35 | 1.36 |
| Average | 46.03 | 106.34 | 0.92 | 1.96 | 1.18 | 0.31 | 12.27 | 2.53 | 25.79 | 357.91 | 20.27 | 1.85 |
| value | | | | | | | | | | | | |
| SD | 6.85 | 14.79 | 0.50 | 0.29 | 0.13 | 0.08 | 1.74 | 0.25 | 6.31 | 43.34 | 4.08 | 0.40 |

Mode group

| | | | | | | | | | | | | |
|---|---|---|---|---|---|---|---|---|---|---|---|---|
| 1 | 91.1 | 270.9 | 0.92 | 1.72 | 1.04 | 0.32 | 16.5 | 6.9 | 86.2 | 180.52 | 50.19 | 1.05 |
| 2 | 75.2 | 260.2 | 1.15 | 1.8 | 0.85 | 0.7 | 18.3 | 5.1 | 106.4 | 200.15 | 39.28 | 0.95 |
| 3 | 75.7 | 204.5 | 1.44 | 2.16 | 1.26 | 0.41 | 18.1 | 5.5 | 94.2 | 185.64 | 45.42 | 0.72 |
| 4 | 95.6 | 185.6 | 1.06 | 2.02 | 1.18 | 0.56 | 17.4 | 5.7 | 77.4 | 200.51 | 58.24 | 0.85 |
| 5 | 85.5 | 216.8 | 1.3 | 2.06 | 1.18 | 0.38 | 17.2 | 6 | 90.1 | 129.67 | 50.29 | 0.96 |
| Average | 84.62 | 227.60 | 1.17 | 1.95 | 1.10 | 0.47 | 17.50 | 5.84 | 90.86 | 179.30 | 48.68 | 0.91 |
| value | | | | | | | | | | | | |
| SD | 9.11 | 36.58 | 0.20 | 0.18 | 0.16 | 0.15 | 0.72 | 0.68 | 10.68 | 29.1 | 6.99 | 0.13 |

High-dose cornus wilsoniana oil group

| | | | | | | | | | | | | |
|---|---|---|---|---|---|---|---|---|---|---|---|---|
| 1 | 40.8 | 146 | 0.68 | 1.76 | 1.16 | 0.28 | 10.2 | 2.7 | 39.2 | 306.22 | 35.13 | 1.72 |
| 2 | 44.4 | 156.7 | 0.93 | 1.85 | 1.1 | 0.32 | 10.4 | 2.5 | 23.2 | 320.86 | 32.45 | 1.17 |
| 3 | 32.9 | 146.1 | 0.77 | 1.46 | 0.93 | 0.25 | 10.5 | 2.9 | 37.6 | 360.95 | 25.54 | 2.16 |
| 4 | 45 | 126.6 | 1.35 | 1.85 | 1.09 | 0.27 | 11 | 2.2 | 33.8 | 301.90 | 27.42 | 1.55 |
| 5 | 41.2 | 155.7 | 1.72 | 1.88 | 0.88 | 0.36 | 14.6 | 2.9 | 29.7 | 320.76 | 39.43 | 1.82 |
| 6 | 51.1 | 159.2 | 1.09 | 2.09 | 1.39 | 0.3 | 12.1 | 2 | 26.2 | 359.74 | 28.32 | 1.77 |
| Average | 42.57 | 148.38 | 1.09 | 1.82 | 1.09 | 0.3 | 11.47 | 2.53 | 31.62 | 328.40 | 31.38 | 1.70 |
| value | | | | | | | | | | | | |
| SD | 6.01 | 12.04 | 0.39 | 0.21 | 0.18 | 0.04 | 1.68 | 0.37 | 6.35 | 25.89 | 5.28 | 0.33 |

Low-dose cornus wilsoniana oil group

| | | | | | | | | | | | | |
|---|---|---|---|---|---|---|---|---|---|---|---|---|
| 1 | 60 | 139.2 | 0.79 | 2.19 | 1.26 | 0.4 | 14.2 | 2.7 | 82 | 220.56 | 40.28 | 1.62 |
| 2 | 72.2 | 136.9 | 0.89 | 2.34 | 1.45 | 0.39 | 15.2 | 3.2 | 79.2 | 170.96 | 45.55 | 1.17 |
| 3 | 60.5 | 160 | 0.8 | 2.12 | 1.16 | 0.48 | 11.6 | 2.6 | 72.1 | 180.49 | 37.74 | 1.06 |
| 4 | 58.3 | 199.2 | 0.84 | 2.19 | 1.2 | 0.45 | 12.6 | 2.5 | 85.3 | 250.57 | 41.38 | 1.68 |
| 5 | 58.9 | 194.9 | 0.93 | 1.9 | 1.23 | 0.28 | 15.5 | 2.8 | 90.1 | 200.32 | 42.35 | 1.16 |
| 6 | 55.7 | 182.2 | 0.63 | 1.87 | 1.25 | 0.29 | 14.9 | 3.4 | 89.5 | 220.83 | 32.67 | 1.22 |
| Average | 60.93 | 168.73 | 0.81 | 2.10 | 1.26 | 0.38 | 14.00 | 2.87 | 83.03 | 207.29 | 40.00 | 1.32 |
| value | | | | | | | | | | | | |
| SD | 5.77 | 27.41 | 0.10 | 0.18 | 0.10 | 0.08 | 1.57 | 0.36 | 6.81 | 29.39 | 4.41 | 0.26 |

1. A method for anti-hepatic fibrosis, comprising a step of administering an extract from Cornus wilsoniana or formulation thereof to an animal in need thereof.

2. The method according to claim 1, wherein the extract from Cornus wilsoniana is Cornus wilsoniana oil.

3. The method according to claim 1, wherein the formulation is a food, a medicament or a health care product.

4. The method according to claim 3, wherein when the formulation is a medicament, the formulation is administered by oral administration, injection or intragastric administration.

5. The method according to claim 4, wherein the medicament is in a form of tablet, capsule, powder-injection, injection or aerosol.

6. The method according to claim 3, wherein when the formulation is a food or a health care product, the formulation further comprises one or more excipients acceptable in the field of galenic pharmacy.

7. The method according to claim 1, wherein the anti-hepatic fibrosis refers to preventing or treating hepatic fibrosis or improving the condition of hepatic fibrosis.

8. The method according to claim 1, wherein the hepatic fibrosis is induced by CCl₄.

9. An anti-hepatic fibrosis formulation, wherein the formulation comprises an extract from Cornus wilsoniana.

10. The formulation according to claim 9, wherein the formulation is Cornus wilsoniana oil.

11. The method according to claim 2, wherein the Cornus wilsoniana oil is a lipid substance extracted from the fruits and seeds of Cornus wilsoniana.

12. The method according to claim 4, wherein the injection is intravenous injection or intraperitoneal injection.

13. The method according to claim 5, wherein the medicament further comprises one or more pharmaceutically acceptable excipients.

14. The method according to claim 6, wherein the formulation further comprises at least one or more of fillers, taste improvers, solvents and buffers.

15. The formulation according to claim 10, wherein the formulation is a food, a medicament or a health care product.

16. The method according to claim 2, wherein the formulation is a food, a medicament or a health care product.

17. The method according to claim 2, wherein the anti-hepatic fibrosis refers to preventing or treating hepatic fibrosis or improving the condition of hepatic fibrosis.

18. The method according to claim 3, wherein the anti-hepatic fibrosis refers to preventing or treating hepatic fibrosis or improving the condition of hepatic fibrosis.

19. The method according to claim 4, wherein the anti-hepatic fibrosis refers to preventing or treating hepatic fibrosis or improving the condition of hepatic fibrosis.

20. The method according to claim 5, wherein the anti-hepatic fibrosis refers to preventing or treating hepatic fibrosis or improving the condition of hepatic fibrosis.
